(19) United States
(12) Patent Application Publication
(10) Pub. No.: US 2025/0295347 A1
(43) Pub. Date: Sep. 25, 2025

(54) COMPUTERIZED ECG INTERPRETATION VISUALIZATION

(71) Applicant: Welch Allyn, Inc., Skaneateles Falls, NY (US)

(72) Inventors: Ryan Donlon, Milton, MA (US); Nicoletta Marzocchi, Bologna (IT); Sharone Zlochiver, Mequon, WI (US); Patrick James Noffke, Oconomowoc, WI (US); Waqaar Khawar, Raleigh, NC (US)

(21) Appl. No.: 19/083,634

(22) Filed: Mar. 19, 2025

Related U.S. Application Data
(60) Provisional application No. 63/568,546, filed on Mar. 22, 2024.

(51) Int. Cl. A61B 5/349; A61B 5/00; A61B 5/339; G16H 70/60

(52) U.S. Cl. CPC A61B 5/349; A61B 5/339; A61B 5/7264; A61B 5/7282; A61B 5/743; A61B 5/7435; A61B 5/748; G16H 70/60

(57) ABSTRACT

An electrocardiogram interpretation system includes a display and a user interface. A processor receives an ECG including a waveform of electrical activity of a patient's heart and analyzes the waveform to calculate computerized interpretation metrics and detect at least one segment of the waveform corresponding to a behavior associated with a condition of the patient's heart and categorize a type of the condition and a status of the condition selected from a group including at least normal and abnormal with the computerized interpretation metrics. The processor generates, on the display, the waveform and highlight the at least one segment with a color selected from a plurality of colors, each color of the plurality of colors corresponding to the type of condition or the status of the condition. The processor generates, on the display, textual information that includes the type of condition or the status of the condition.

### CROSS-REFERENCE TO RELATED APPLICATION

This application claims priority under U.S.C. § 119(e) to U.S. Provisional Application No. 63/568,546 filed on Mar. 22, 2024, entitled “COMPUTERIZED ECG INTERPRETATION VISUALIZATION,” the disclosure of which is hereby incorporated by reference in its entirety.

### FIELD OF THE DISCLOSURE

The present disclosure generally relates to an electrocardiogramaualization system, and, more particularly, a visualization system that assists a caregiver in interpreting an electrocardiogram by highlighting points of interest.

### BACKGROUND

Computerized electrocardiogramterpretation has been in development for several decades. As technology advances, various devices have been developed with computerized interpretation (“CI”), such as electrocardiogram, which analyze the ECG and present measurements (e.g., HR, PR, QRS duration, QT/QTc) and textual interpretation statements. The results of the CI are unconfirmed, assistive in nature, and must be reviewed by a physician in order for them to be clinically meaningful. In practice, however, physicians may rely on the CI to make clinical decisions. As such, it is important that a physician understand the basis of the CI in order to accurately and independently make a clinically meaningful diagnosis.

Accordingly, there is a continued need to make not only the CI understandable to physicians, but also the basis for the CI, such that the physician can accurately and independently form a diagnosis.

### SUMMARY OF THE DISCLOSURE

According to one aspect of the present disclosure, an electrocardiogram (“ECG”) interpretation system includes a display, a user interface, a processor, and a memory. The memory contains instructions that, when executed by the processor, cause the processor to receive an ECG that includes a waveform of electrical activity of a patient's heart over a period of time and analyze the waveform to calculate computerized interpretation metrics. The processor is further caused to detect at least one segment of the waveform corresponding to a behavior associated with a condition of the patient's heart and categorize a type of the condition and a status of the condition selected from a group including at least normal and abnormal with the computerized interpretation metrics. The processor is further caused to generate, on the display, the waveform and highlight the at least one segment with a color selected from a plurality of colors, each color of the plurality of colors corresponding to the type of condition or the status of the condition. The processor is further caused to generate, on the display, textual information that includes the type of condition or the status of the condition.

According to another aspect of the present disclosure, an electrocardiogram (“ECG”) interpretation system includes a display, a user interface, a processor, and a memory. The memory contains instructions that, when executed by the processor, cause the processor to receive an ECG that includes a waveform of electrical activity of a patient's heart over a period of time and analyze the waveform to calculate computerized interpretation metrics. The processor is further caused to detect at least one segment of the waveform corresponding to an abnormality of the patient's heart with the computerized interpretation metrics. The processor is further caused to generate, on the display, the waveform, and generate on the display, textual information that includes the type of the abnormality and highlight the textual information with a color selected from a plurality of colors, each color of the plurality of colors corresponding to the severity of the abnormality.

According to yet another aspect of the present disclosure, an electrocardiogram (“ECG”) interpretation system includes a display, a user interface, a processor, and a memory. The memory contains instructions that, when executed by the processor, cause the processor to receive an ECG that includes a waveform of electrical activity of a patient's heart over a period of time and analyze the waveform to calculate computerized interpretation metrics. The processor is further caused to detect at least one segment of the waveform corresponding to a behavior associated with a condition of the patient's heart and categorize a type of the condition and a status of the condition selected from a group including at least normal and abnormal with the computerized interpretation metrics. The processor is further caused to generate, on the display, the waveform and highlight the at least one segment with a color selected from a plurality of colors, each color of the plurality of colors corresponding to the type of condition or the status of the condition. The processor is further caused to generate, on the display, textual information that includes the type of condition or the status of the condition and highlight the textual information with the color selected from the plurality of colors, each color of the plurality of colors corresponding to the type of condition or the status of the condition.

According to still yet another aspect of the disclosure, an electrocardiogram (“ECG”) interpretation system includes a display, a user interface, a processor, and a memory. The memory contains instructions that, when executed by the processor, cause the processor to receive an ECG that includes a waveform of electrical activity of a patient's heart over a period of time and analyze the waveform to calculate computerized interpretation metrics. The processor is further caused to detect at least one segment of the waveform corresponding to a behavior associated with a condition of the patient's heart and categorize a type of the condition and a status of the condition selected from a group including at least normal and abnormal with the computerized interpretation metrics. The processor is further caused to generate, on the display, the waveform and highlight the at least one segment with a first color selected from a plurality of first colors, each first color corresponding to the type of condition or the status of the condition. The processor is further caused to generate, on the display, textual information that includes the type of condition or the status of the condition and highlight the textual information with the first color selected from the first plurality of colors, each color of the plurality of colors corresponding to the type of condition or the status of the condition.

According to still another aspect of the disclosure, an electrocardiogram (“ECG”) interpretation system includes an electrocardiogram device configured to place over a patient's heart that generates a cardiac signal. The ECG interpretation system further includes a display, a user interface, a processor, and a memory. The memory contains instructions that, when executed by the processor, cause the processor to receive, from the electrocardiogram device, the cardiac signal and generate an ECG that includes a waveform of electrical activity of a patient's heart over a period of time, and analyze the waveform to calculate computerized interpretation metrics. The processor is further caused to detect at least one segment of the waveform corresponding to a behavior associated with a condition of the patient's heart and categorize a type of the condition and a status of the condition selected from a group including at least normal and abnormal with the computerized interpretation metrics. The processor is further caused to generate, on the display, the waveform and highlight the at least one segment with a color selected from a plurality of colors, each color of the plurality of colors corresponding to the type of condition or the status of the condition. The processor is further caused to generate, on the display, textual information that includes the type of condition or the status of the condition.

According to still yet another aspect, a method of interpreting an electrocardiogram (“ECG”) includes receiving an ECG that includes a waveform of electrical activity of a patient's heart over a period of time. The waveform is analyzed to calculate computerized interpretation metrics and at least one segment of the waveform is detected that corresponds to a behavior associated with a condition of the patient's heart. The method further includes categorizing a type of the condition and a status of the condition selected from a group including at least normal and abnormal with the computerized interpretation metrics, and generating, on a display, the waveform and highlight the at least one segment with a color selected from a plurality of colors, each color of the plurality of colors corresponding to the type of condition or the status of the condition. On the display, textual information is generated that includes the type of the-condition and the status of the condition.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a partially schematic view of an electrocardiogram interpretation system that includes a control system, a display, and an electrocardiogram device, according to the present disclosure;

FIG. 2 is a schematic view of a control system of an electrocardiogram interpretation system, according to the present disclosure;

FIG. 3 is a first enlarged image on a display generated by a control system of an electrocardiogram interpretation system, according to the present disclosure;

FIG. 4 is a second enlarged image on a display generated by a control system of an electrocardiogram interpretation system, according to the present disclosure;

FIG. 5 is a third enlarged image on a display generated by a control system of an electrocardiogram interpretation system, according to the present disclosure;

FIG. 6 is a fourth enlarged image on a display generated by a control system of an electrocardiogram interpretation system, according to the present disclosure;

FIG. 7 is a fifth enlarged image on a display generated by a control system of an electrocardiogram interpretation system, according to the present disclosure;

FIG. 8 is an image on a display generated by a control system of an electrocardiogram interpretation system, according to the present disclosure;

FIG. 9 is a first partial image on a display generated by a control system of an electrocardiogram interpretation system, according to the present disclosure;

FIG. 10 is a second partial image on a display generated by a control system of an electrocardiogram interpretation system, according to the present disclosure;

FIG. 11 is a third partial image on a display generated by a control system of an electrocardiogram interpretation system, according to the present disclosure;

FIG. 12 is a fourth partial image on a display generated by a control system of an electrocardiogram interpretation system, according to the present disclosure; and

FIG. 13 is a flow chart showing a method of visualizing a computerized interpretation on a display with a user interface, according to the present disclosure.

### DETAILED DESCRIPTION

The present illustrated embodiments reside primarily in combinations of method steps, systems, devices, and apparatus components related to a visualization system that assists a caregiver in interpreting an electrocardiogram by highlighting points of interest. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

The specific structures and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

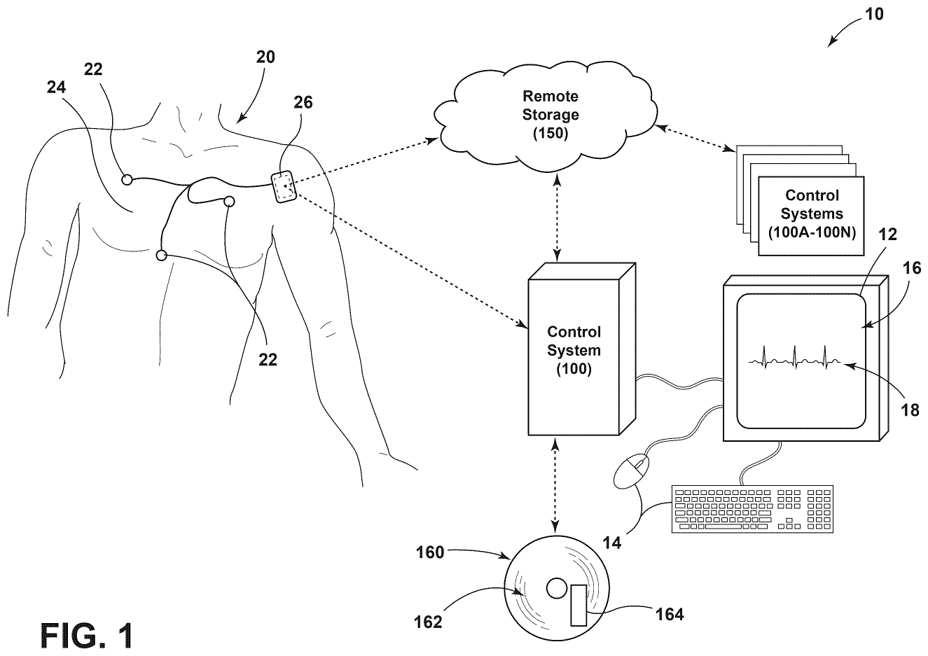

Referring initially to FIGS. 1-12, reference numeral 10 designates an electrocardiogram (“ECG”) interpretation system. The ECG interpretation system 10 includes a display 12, a user interface 14, a control system 100 including a processor 104, and a memory 106. The memory 106 contains instructions that, when executed by the processor 104, cause the processor 104 to receive an ECG 16 that includes a waveform 18 of electrical activity of a patient's heart over a period of time and analyze the waveform 18 to calculate computerized interpretation metrics. The processor 104 then detects at least one segment S1 of the waveform 18 corresponding to a behavior associated with a condition of the patient's heart and categorizes the type of condition or the status of the condition with the computerized interpretation metrics. The processor 104 then generates, on the display 12, the waveform 18 and highlights the at least one segment S1 with a color selected from a plurality of colors, each color of the plurality of colors corresponding to the type of condition or the status of the condition. The processor 104 further generates, on the display 12, textual information “Ti” that includes the type of condition or the status of the condition.

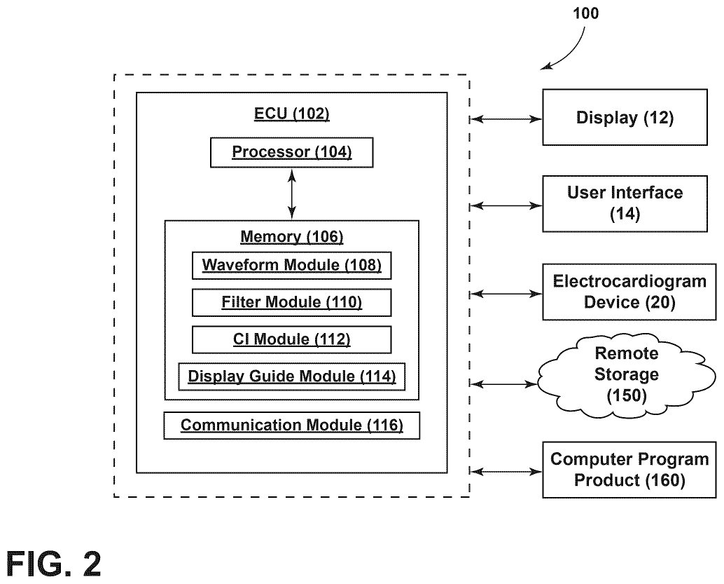

Referring now to FIGS. 1 and 2, the ECG interpretation system 10 may receive the ECG 16 from an electrocardiogram device 20 directly or indirectly. For example, in some embodiments, the ECG interpretation system 10 (e.g., the control system 100) may be configured to receive, from the electrocardiogram device 20, a cardiac signal and generate the ECG 16 that includes the waveform 18 of electrical activity of the patient's heart. The electrical activity of the patient's heart may correspond to cardiac muscle activity, non-cardiac muscle activity, motion artifacts, combinations thereof, and/or the like. In such embodiments, the comparison to the computerized interpretation metrics may occur in real-time and/or near real-time for monitoring a current condition of the patient's heart. Further in such embodiments, the controller 100 may be configured to control functionality of both the electrocardiogram device 20 (i.e., the cardiac signal generating component) and the computerized interpretation components. However, in other embodiments, the electrocardiogram device 20, or a computing device different than the electrocardiogram device 20 and the controller 100, may be configured to generate the ECG 16 from the cardiac signal that is stored remotely or locally which can later be received by the control system 100 (e.g., memory 106) for comparison to the computerized interpretation metrics.

With reference to FIG. 1, the electrocardiogram device 20 may be configured to place over the patient's heart. More particularly, the electrocardiogram device 20 may include a plurality of electrodes 22 placed on a patient's chest 24 proximate the patient's heart for generating the cardiac signal that is used to generate the waveform 18 over a period of time. The electrocardiogram device 20 may include a communication device 26 in communication with at least one of the control system 100 and the remote storage 150. The communication may be wired or wireless. It should be appreciated that the electrocardiogram device 20 depicted in FIG. 1 is exemplary only, electrocardiogram devices of different configurations may be utilized for receiving and/or generating the cardiac signal. For example, electrocardiogram devices that are mobile, stationary, and operate under various principles with different configurations of electrodes may be used other than the electrocardiogram device 20 depicted in FIG. 1. For example, in some embodiments, the electrodes 22 may be in a 12-lead configuration. In use, the behavior (e.g., electrical activity) of the patient's heart generates the cardiac signal, which is transmitted to the control system 100. The control system 100 may store the cardiac signal locally and/or in a remote storage 150, such as a cloud or a remote server. The remote storage 150 may be operably connected to the control system 100 and/or numerous additional control systems 100A-100N located within one or more medical environments (e.g., a hospital, a lab, or a medical campus) locally to the electrocardiogram device 20, regionally, nationally, or internationally. The control system 100 accesses the cardiac signal (e.g., electrical activity) to generate the waveform 18 over the period of time and analyzes the waveform 18 to calculate computerized interpretation metrics, wherein different features can be accessed via the display 12. The display 12 may be configured as a computer, a tablet, and/or the like and include a user interface 14. The user interface 14 may include a touchscreen, a keypad (e.g., a keyboard), a mouse and/or the like for interacting with the visualization on the display 12.

FIG. 2 schematically illustrates the control system 100. In the depicted implementation, the control system 100 may include an electronic control unit (ECU) 102. The ECU 102 may include the processor 104 and the memory 106. The processor 104 may include any suitable processor 104. Additionally, or alternatively, the ECU 102 may include any suitable number of processors, in addition to or other than the processor 104. The memory 106 may comprise a single disk or a plurality of disks (e.g., hard drives) and includes a storage management module that manages one or more partitions within the memory 106. In some embodiments, memory 106 may include flash memory, semiconductor (solid-state) memory, or the like. The memory 106 may include Random Access Memory (RAM), a Read-Only Memory (ROM), Electrically Erasable Programmable Read-Only Memory (EEPROM), any type of non-transitory memory, or a combination thereof. The memory 106 may include instructions that, when executed by the processor 104, cause the processor 104 to, at least, perform the methods and functions associated with the components of the control system 100. As will be appreciated with further reading, the control system 100 may in addition or alternatively be associated with and/or receive instructions from a computer program product 160 that includes instructions to carry out the methods and functionalities described herein. In this manner, in some implementations, the memory 106 may include at least some instructions received from the computer program product 160. The electrocardiogram device 20, the display 12, and the user interface 14 may therefore be controlled and/or receive instructions from the ECU 102. In some embodiments, the electrocardiogram device 20 may include a control system different than the control system 100 that saves information directly and/or remotely through an intermediary (e.g., the remote storage 150) onto memory 106. The memory 106 may therefore include a waveform module 108, a filter module 110, a computerized interpretation module 112 (“CI module”), and a display guide module 114. The control system 100 may further include a communication module 116 that receives/transmits information with the remote storage 150 and/or the electrocardiogram device 20.

With continued reference to FIG. 2, the control system 100 is configured to receive at least one of the cardiac signal or the waveform 18 over the period of time from the electrocardiogram device 20 directly or from the remote storage 150. When only the cardiac signal is received, the waveform module 108 may generate the waveform 18 and save the waveform in memory 106 (e.g., in the waveform module 108). When the waveform 18 is received from electrocardiogram device 20 or the remote storage 150, the waveform 18 may be stored in the waveform module 108. Once the waveform 18 is received. The filter module 110 may detect anomalies from the waveform 18 (i.e., incorrect electrode 22 placement, shifting of the patient's chest 24, etc.). The anomalies may still be shown in the waveform 18 but the control system 100 (e.g., the processor 104) may generate a textual note or other indicia so that a healthcare provider can make a final judgment on whether to consider the anomalies in the clinical diagnosis. The waveform 18 is then compared to the computerized interpretation metrics, which may be located in the computerized interpretation module 112 and/or in the remote storage 150. The computerized interpretation module 112 may include a library of predictive models, threshold values, and other information metrics related to a normal waveform 18, including threshold values of the waveform 18 corresponding to the type of condition or the status of the condition (e.g., a severity and/or abnormality type). More particularly, the computerized interpretation module 112 may operate under the principles of various commercial products that provide computerized interpretation metrics. Based on the comparison between the waveform 18 and computerized interpretation metrics, the control system 100 (e.g., the processor 104 receiving instructions from the computerized interpretation module 112) is further caused to detect at least one segment S1 of the waveform corresponding to the type of condition or the status of the condition of the patient's heart and categorize the type of condition or the status of the condition. The control system 100 (e.g., the processor 104 receiving instructions from the display guide module 114) is further caused to generate, on the display 12, the waveform 18 and highlight the at least one segment S1 with the color. The display guide module 114 may include the plurality of colors, with each color of the plurality of colors corresponding to the type of condition or the status of the condition (e.g., the presence and severity of an abnormality or normal behavior). The control system 100 (e.g., the processor 104 receiving instructions from the display guide module 114) is further caused to generate, on the display 12, textual information Ti that includes the type of condition or the status of the condition.

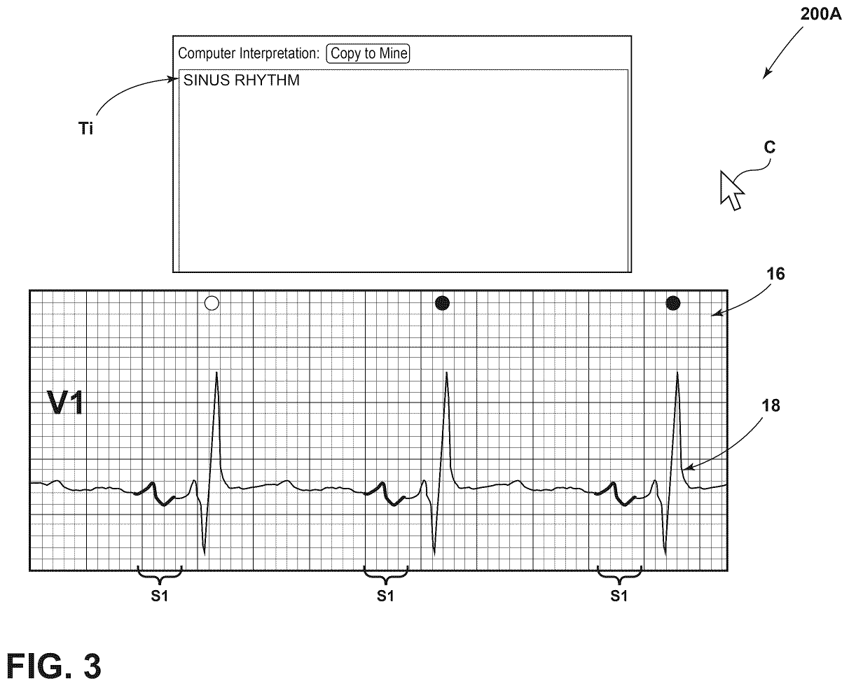

With continued reference to FIG. 2, in one example, the aforementioned computer program product 160 may include many of the instructions associated with the memory 106. More particularly, the instructions included in the computer program product 160 may include instructions (e.g., saved in non-transitory memory) related to creating and generating the different graphics described herein and shown in FIGS. 3-12, including functions associated therewith. More particularly, the computer program product 160 may link information between the display 12, the user interface 14, the electrocardiogram device 20, and/or devices associated therewith. For example, the computer program product 160 may include instructions to perform the method steps and functions described herein, but may rely on certain features/inputs such as from the one or more user interface 14. Further, select or all features of the filter module 110, a computerized interpretation module 112 (“CI module”), and a display guide module 114 may be contained the computer program product 160.

The computer program product 160 may include, for instance, one or more computer-readable medium 162 (e.g., non-transitory memory) to store computer-readable program code means or logic 164 in order to provide and facilitate one or more functions and methods steps described in the present disclosure. The program code contained or stored in/on a computer- readable medium 162 can be obtained and executed by a computer, such as the control system 100 to behave/function/generate instructions in a particular manner. The program code can be transmitted using any appropriate medium, including (but not limited to) wireless, wireline, optical fiber, and/or radio-frequency. The program code 164 includes instructions for carrying out operations to perform, achieve, or facilitate aspects of the disclosure may be written in one or more programming languages. In some embodiments, the programming language(s) include object-oriented and/or procedural programming languages such as C, C++, C #, Java, and/or the like. However, it should be appreciated that the control system 100 without the computer program product 160 may perform all the functions, steps, and aspects of the disclosure, such as those described in reference to FIGS. 3-12.

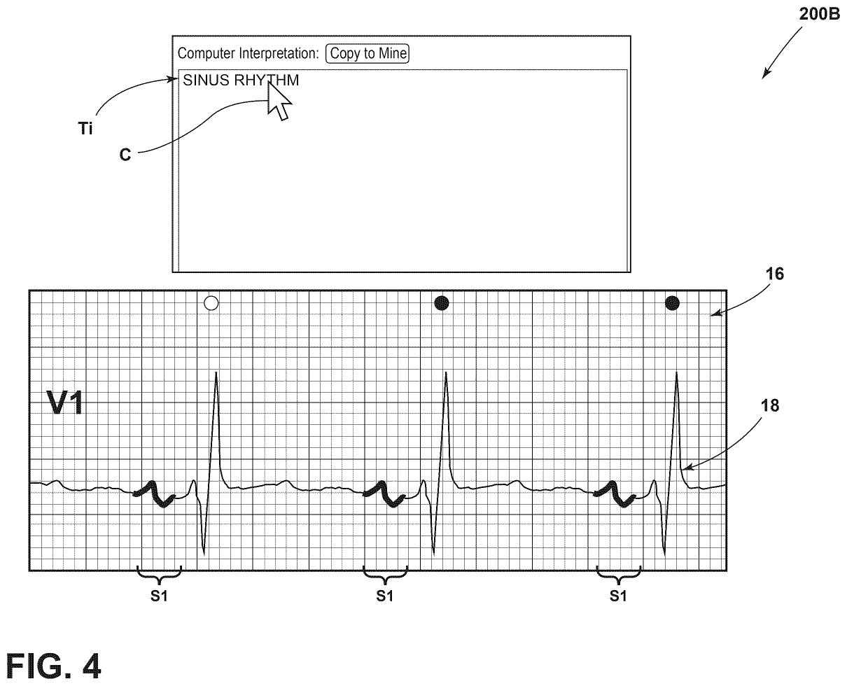

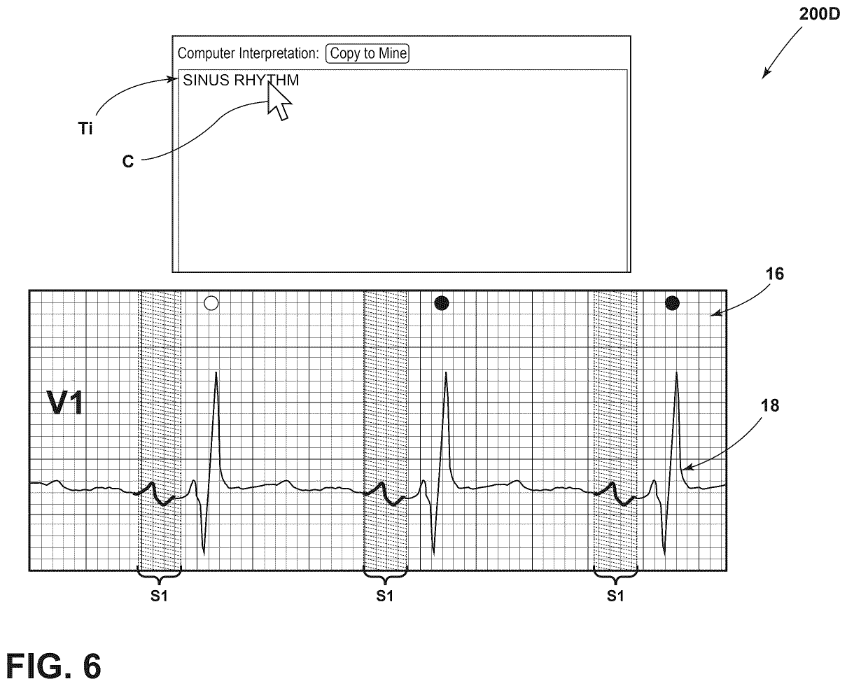

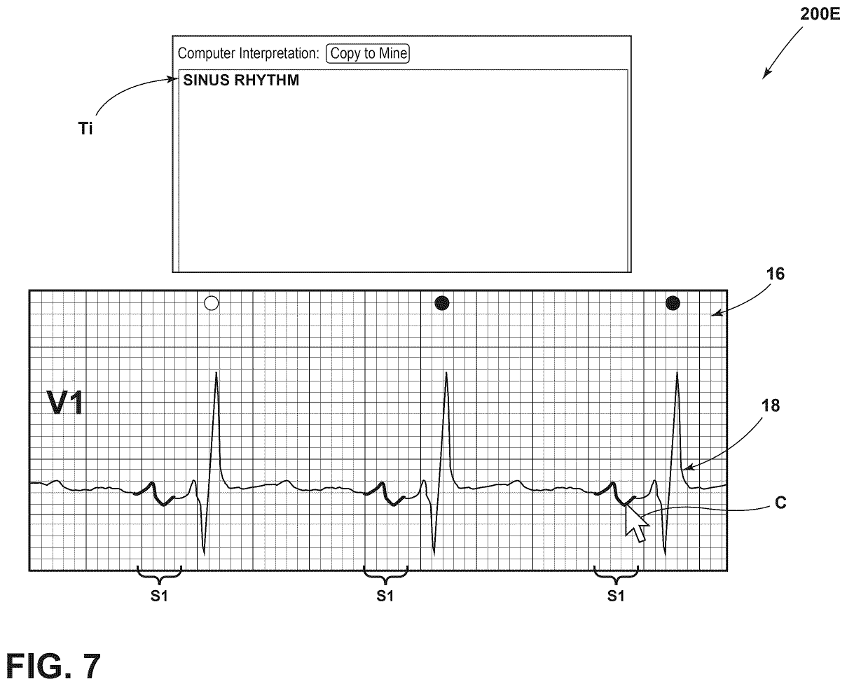

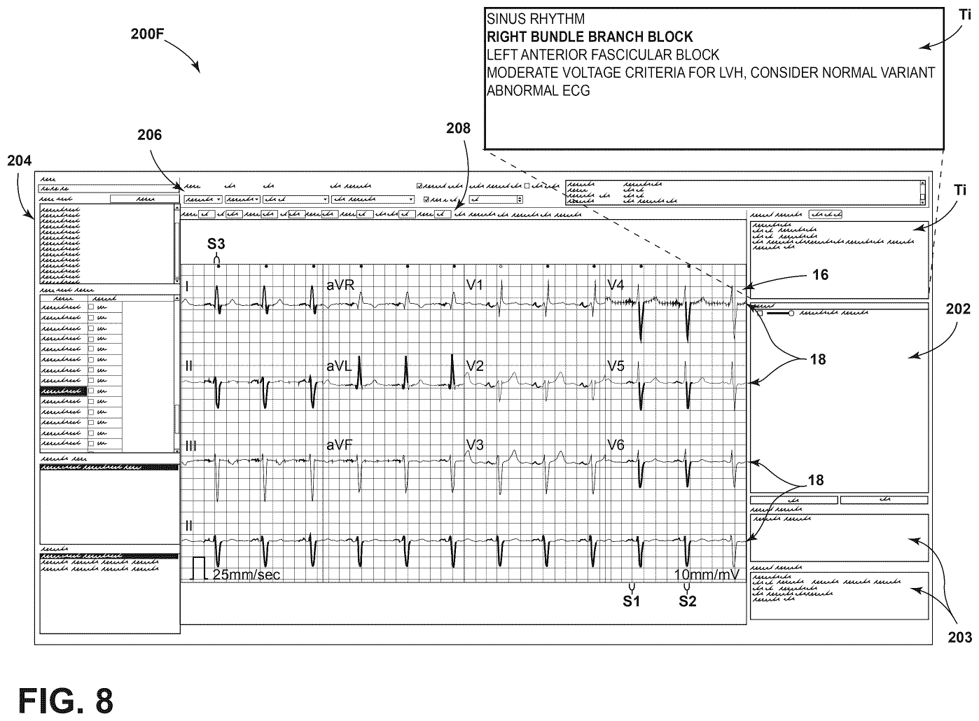

FIGS. 3-12 illustrate various examples of the images 200A-200J generated on the display 12. However, it should be appreciated that the images 200A-200J are exemplary in nature, and a variety of alternative ways of visualizing important information could be utilized. For example, the term “highlight” may be defined as a generation of an indicia on the image 200A-200J to draw the healthcare provider's attention to the at least one segment S1 (e.g., that is abnormal) and to visualize the type of condition or the status of the condition associated with the at least one segment S1. The highlight may include selecting a color out of a plurality of colors based on severity. The plurality of colors may be different than non-highlighted segments for easy visualization and interpretation. In some embodiments, the color may be generated on the segment S1, such that the waveform 18 is the color. In some embodiments, the color may be generated on a background behind the segment S1 (FIG. 6). Likewise, a matching highlight (e.g., the same color) may be generated relative to the textual information Ti. In some embodiments, the color may be generated on the textual information, such that font is the color. In some embodiments, the color may be generated on a background behind the textual information. The highlight may include other types of indicia, for example, the highlight may include bolding/patterning the waveform 18 (e.g., with dotted lines, dashed lines, thickened lines, and/or the like) or the font of the textual information Ti (FIGS. 4 and 7). In some embodiments, two or more highlights may be utilized (FIGS. 8-12). For example, a first type of highlight corresponding to the type of condition and a second type of highlight corresponding to the status of the condition (e.g., abnormality presence and severity). In some embodiments, the two highlights may be applied to the segment S1 and/or the textual information Ti. In still other embodiments, the first highlight may be generated on one of the segments S1-SN or the textual information Ti and the second highlight may be generated on the other of the segment S1-SN and the textual information Ti. In addition to the highlight, it should be appreciated that the textual information Ti may include the type and or status of the condition in written form.

With continued reference to FIGS. 1-12, in some embodiments, the control system 100 (e.g., the processor 104) may be configured to, based on instructions in the memory 106, the remote storage 150, and/or the computer program product 160, receive an ECG 16 that includes a waveform 18 of electrical activity of a patient's heart over a period of time. For example, the ECG 16 may be received from the electrocardiogram device 20 or from the remote storage 150 and may further be stored in memory 106 (i.e., the waveform module 108).

Once the ECG has been received, in some embodiments, the control system 100 (e.g., the processor 104) may be configured to, based on instructions in the memory 106, the remote storage 150, and/or the computer program product 160, detect anomalies (i.e., with the filter module 110) from the waveform 18 (i.e., incorrect electrode 22 placement, shifting of the patient's chest 24, etc.). The anomalies may still be shown in the waveform 18 but the control system 100 (e.g., the processor 104) may generate a textual note or other indicia (e.g., the highlight) so that a healthcare provider can make a final judgment on whether to consider the anomalies in the clinical diagnosis. It should be appreciated that, in some embodiments, the filtering operation by the filtering module 110 may be included in the computerized interpretation module 112 (e.g., with predictive models, threshold information, and/or the like).

In some embodiments, the control system 100 (e.g., the processor 104) may be configured to, based on instructions in the memory 106, the remote storage 150, and/or the computer program product 160, analyze the waveform 18 to calculate computerized interpretation metrics. The computerized interpretation module 112, the remote storage 150, and/or the computer product 160, may include a library of predictive models, threshold values, and other information metrics related to a normal waveform 18, including threshold values of the waveform 18 corresponding to the type of condition or the status of the condition.

With continued reference to FIGS. 3-12, in some embodiments, the control system 100 (e.g., the processor 104) may be configured to, based on instructions in the memory 106, the remote storage 150, and/or the computer program product 160, detect at least one segment S1 of the waveform 18 corresponding to a behavior associated with a condition of the patient's heart and categorize the type of condition or the status of the condition with the computerized interpretation metrics. For example, the computerized interpretation module 112, the remote storage 150, and/or the computer program product 160 may include a library of predictive models, threshold values, and other information metrics related to a normal waveform 18, including threshold values of the waveform 18 corresponding to the type of condition or the status of the condition.

With reference now to FIGS. 3-7, an enlarged view of various portions of images 200A-200E on a display 12 are illustrated to depict how the conveyance of information is streamlined to a healthcare professional. In some embodiments, the control system 100 (e.g., the processor 104) may be configured to, based on instructions in the memory 106 (i.e., the display guide module 114), the remote storage 150, and/or the computer program product 160, generate on the display 12, the waveform 18 and highlight the at least one segment S1 with a color selected from a plurality of colors, each color of the plurality of colors corresponding to the type of condition or the status of the condition that may be different than non-highlighted segments. The processor 104 is further caused to generate, on the display 12, textual information “Ti” that includes the type of the condition. For example, the enlarged image 200A depicted in FIG. 3 shows the waveform 18 and the highlight to the at least one segment S1 and the textual information Ti. In some embodiments, the computerized interpretation metrics in the computerized interpretation module 112 may include different types of highlights (i.e., color, line type, pattern, font type) corresponding to different levels of the status of the condition. The textual information Ti may include information related to portions of the waveform 18 other than the at least one segment S1. Indeed, it should be appreciated that the at least one segment S1 could include a plurality of segments S1-SN corresponding to the same detected type of condition or multiple different types of conditions. In instances with detected multiple different types of conditions, the highlight may, in addition to visualizing status (e.g., severity), visualize the type of condition (i.e., with two or more of the plurality of colors, the textual information Ti, line type, line size, font size, and/or the like). As will be appreciated with further reading, the highlight may be automatically generated on the display 12 or may otherwise only be generated based on inputs from the user interface 14. In some embodiments, the textual information Ti that includes the type of the condition is highlighted in the color of the highlight to the at least one segment S1.

With continued reference to FIGS. 3-7, in some embodiments, the control system 100 (e.g., the processor 104) may be configured to, based on instructions in the memory 106 (i.e., the display guide module 114), the remote storage 150, and/or the computer program product 160, highlight the waveform 18 (e.g., the at least one segment S1) by colorizing the at least one segment in the color, such as the waveform 18 in the enlarged image 200A depicted in FIG. 3. In some embodiments, the highlight on the textual information includes colorizing the font of the textual information Ti in the color (i.e., the same color as the segment S1).

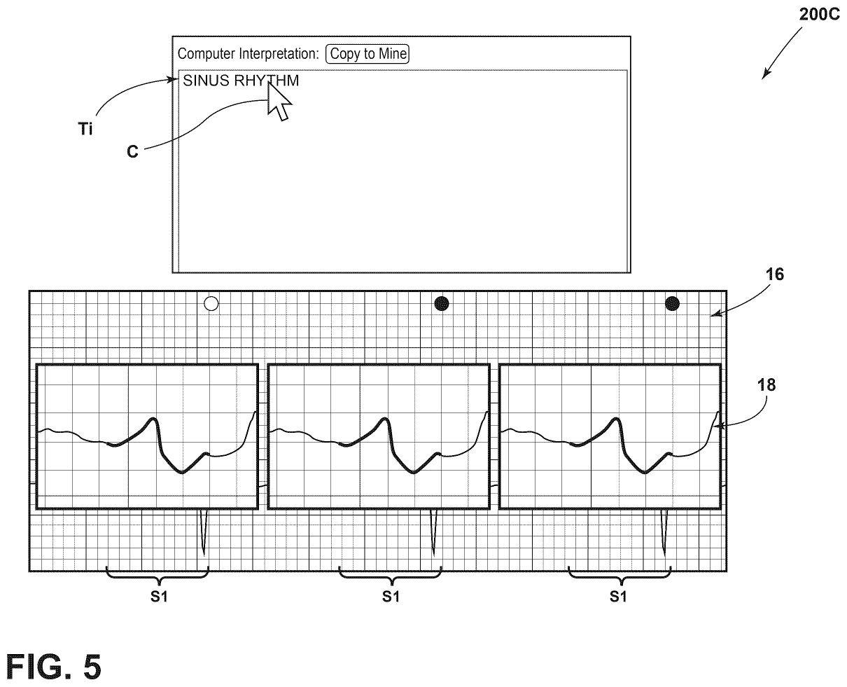

With reference now to FIGS. 4-6, in some embodiments, the user interface 14 includes a cursor “C” and the control system 100 (e.g., the processor 104) may be configured to, based on instructions in the memory 106 (i.e., the display guide module 114), the remote storage 150, and/or the computer program product 160, detect if the cursor is aligned with the textual information Ti that includes the type of the condition and enlarge the at least one segment S1 on the display 12. The cursor C may be a visible graphical symbol, such as the depicted arrow. However, in some implementations, the cursor C may not be graphically visible, such as in situations where the display 12 and user interface 14 are operating via touchscreen technology. In this manner, the term cursor may be a visible graphical symbol or simply a location of the display 12 that is interfaced with by a user via the user interface 14 (e.g., a touch input). For example, in the waveform 18 in enlarged image 200B depicted in FIG. 4, when the cursor C is aligned with the textual information Ti and/or the textual information Ti is otherwise interfaced with, the at least one segment S1 is thickened. Similarly, in the waveform 18 in enlarged image 200C depicted in FIG. 5, when the cursor C is aligned with the textual information Ti and/or the textual information Ti is otherwise interfaced with, the at least one segment S1 is zoomed in on. In cases where there are numerous segments S1-SN associated with the type of condition and/or the status of the condition, each or select segments S1-SN may be zoomed in on and/or thickened. In this manner, a healthcare provider reviewing the ECG 16 may quickly see which segments S1-SN formed the basis for the categorization of the type of condition or the status of the condition with the computerized interpretation metrics in order to make a clinical diagnosis. In other embodiments, such as the waveform 18 in the enlarged image 200D depicted in FIG. 6, the segment S1 may be highlighted by colorizing a background of the segment S1. Likewise, in other embodiments, the textual information Ti may be highlighted by colorizing a background behind the font.

With reference now to FIG. 7, in some embodiments, the user interface 14 includes the cursor C and the control system 100 (e.g., the processor 104) may be configured to, based on instructions in the memory 106 (i.e., the display guide module 114), the remote storage 150, and/or the computer program product 160, detect if the cursor on the enlarged image 200E is aligned with the at least one segment S1 and/or the segment S1 is otherwise interfaced with and enlarge textual information Ti that includes the type of the condition. For example, the textual information Ti may be bolded and/or provided in a larger font. In this manner, a healthcare provider reviewing the ECG 16 may quickly see which segments S1-SN correspond to the textual information Ti in order to make a clinical diagnosis.

With continued reference to the image 200E depicted in FIG. 7, in some embodiments, the control system 100 (e.g., the processor 104) may be configured to, based on instructions in the memory 106 (i.e., the display guide module 114), the remote storage 150, and/or the computer program product 160, initially generate, on the display 12, textual information Ti that includes the type of the condition and highlight the textual information Ti with a color selected from a plurality of colors, rather than or in addition to the segments S1-SN. In some embodiments, the segment S1-SN is not initially colored until the specific textual information Ti is selected with the user interface 14 (e.g., the cursor C), where the associated segments S1-SN become colored. In some embodiments, both the textual information Ti and the segments S1-SN are initially covered. In some embodiments, only the segments S1-SN are initially colored, and the textual information Ti is not colored or becomes colored based on the selection of the segment S1-SN with the user interface 14 (e.g., the cursor).

With reference now to FIGS. 8-12, the highlight may include the plurality of colors as previously described. The plurality of colors may include a first color assigned to normal and a second color assigned to abnormal. Further, the plurality of colors may include a third color assigned to borderline, a fourth color assigned to critical, and a fifth color assigned to abnormal rhythm. In some embodiments, the color scheme includes changes in hue where a soft color such as blue, purple, and/or green is associated with less severity and a harder color such as yellow, orange, and/or red is associated with more severity. In some embodiments, portions of the waveform 18 assigned normal may be in black or dark blue to contrast the different colors assigned to abnormalities of varying severity. Each segment S1-SN may be associated with the type of condition. In this manner, when the textual information Ti is highlighted relating to one particular type of condition, the segment S1-SN associated with that type of condition may likewise be highlighted and/or otherwise enlarged, while the other segments S1-SN are unchanged. As such, the healthcare provider's attention is drawn to the particular segment S1-SN that formed the basis of the computerized interpretation related in the textual information Ti.

With reference now to FIG. 8, an image 200F is depicted illustrating different features that may be generated on the display 12. For example, in some embodiments, the user interface 14 includes a keypad and the control system 100 (e.g., the processor 104) may be configured to, based on instructions in the memory 106 (i.e., the display guide module 114), the remote storage 150, and/or the computer program product 160, generate a diagnosis section 203 that receives inputs and/or comments from the keypad from a healthcare provider related to a clinical diagnosis. More particularly, the healthcare provider may be able to quickly visualize the basis for the computerized interpretation and formulate the clinical diagnosis within the diagnosis section 202 of the image 200A-200J. In still further embodiments, a healthcare provider may be able to interface with the textual information Ti (e.g., via the cursor, a touchscreen, and/or the like) and select an option to incorporate portions of the computerized interpretation into the diagnosis section 202.

With continued reference to FIG. 8, the image 200F generated on the display 12 may further include a notes section 203 for the healthcare provider to enter notes during formulation of the clinical diagnosis, a record section 204, which may include a series of additional ECG 16 or waveforms 18 the same patient (e.g., the patient's history) or other patients. In this manner, the healthcare provider can quickly review numerous ECGs 16. A tool bar 206, with features such as adjusting the values along different axes of the ECG 16 may also be utilized. Further, the image 200F generated on the display 12 may include actual measured values of various heart behaviors in value section 208. The value section 208 may be changed depending on the alignment of the cursor C and/or otherwise interfacing with the waveform 18, a selection of the textual information Ti, and/or the like. The image 200F includes three noteworthy abnormalities communicated by the textual information Ti. These abnormalities include sinus rhythm as shown with segment S1, a right bundle branch block as shown with segment S2, and a left anterior fascicular block as shown with segment S3. The textual information Ti associated with each of these abnormalities is generated in a different color that corresponds to severity. Likewise, the segments S1-S3 associated with each of these abnormalities is generated in the same color as the associated textual information Ti. In the image 200F, the textual information Ti that recites the right bundle branch block or the segment S2 has been selected (e.g., via the user interface). As a result, the segment S2 of the waveform 18 has been enlarged (e.g., thickened).

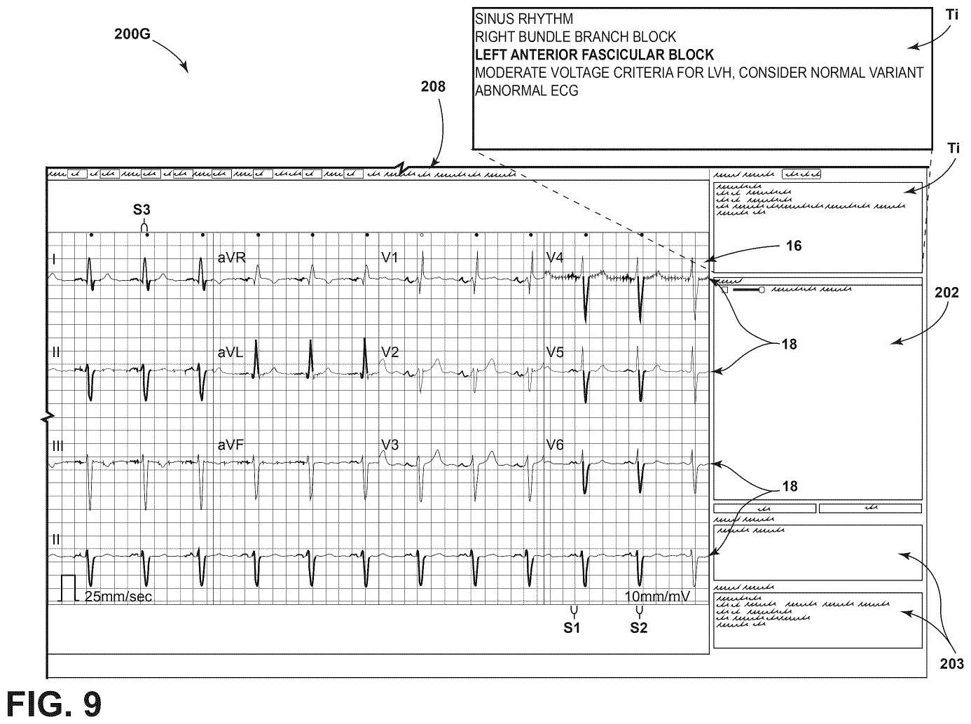

With reference now to FIG. 9, a first partial image 200G generated on the display 12 is illustrated. The first partial image 200G includes the ECG 16 depicted in FIG. 8, but with different features removed for simplicity and a different condition selected. More particularly, in the first partial image 200G, the textual information Ti that recites the left anterior fascicular block or the segment S3 has been selected (e.g., via the user interface). As a result, the segment S3 of the waveform 18 has been enlarged (e.g., thickened).

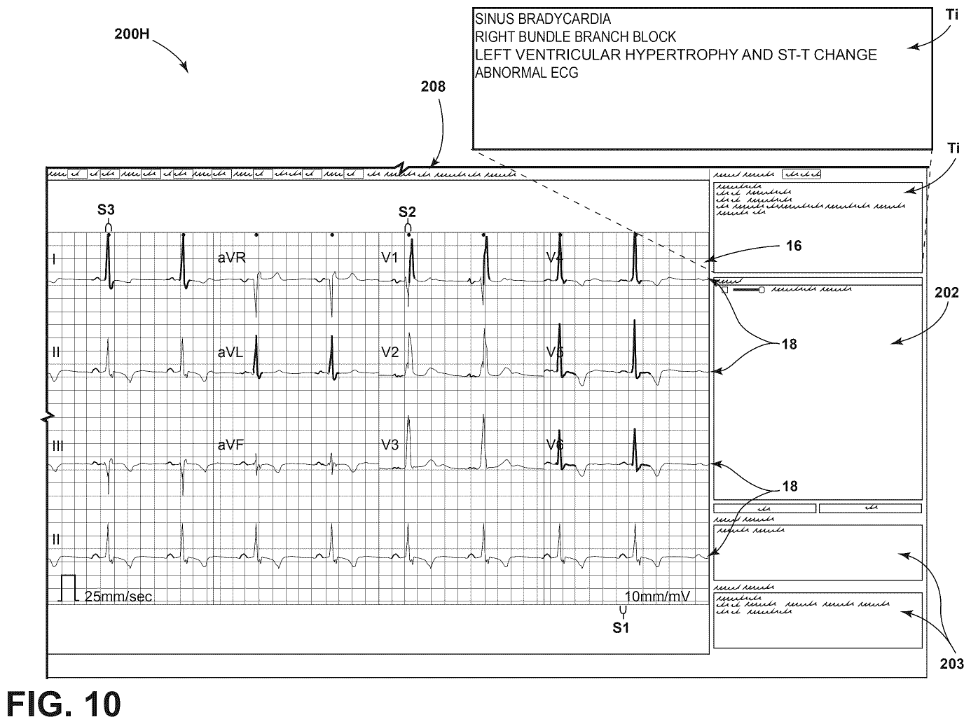

With reference now to FIGS. 10-12, a series of partial images 200H-200J are illustrated. The partial images 200H-200J include three noteworthy abnormalities communicated by the textual information Ti. These abnormalities include sinus bradycardia as shown with segment S1, a right bundle branch block as shown with segment S2, and a left ventricular hypertrophy and ST-T change as shown with segment S3. The textual information Ti associated with each of these abnormalities is generated in a different color that corresponds to severity. Likewise, the segments S1-S3 associated with each of these abnormalities is generated in the same color as the associated textual information Ti.

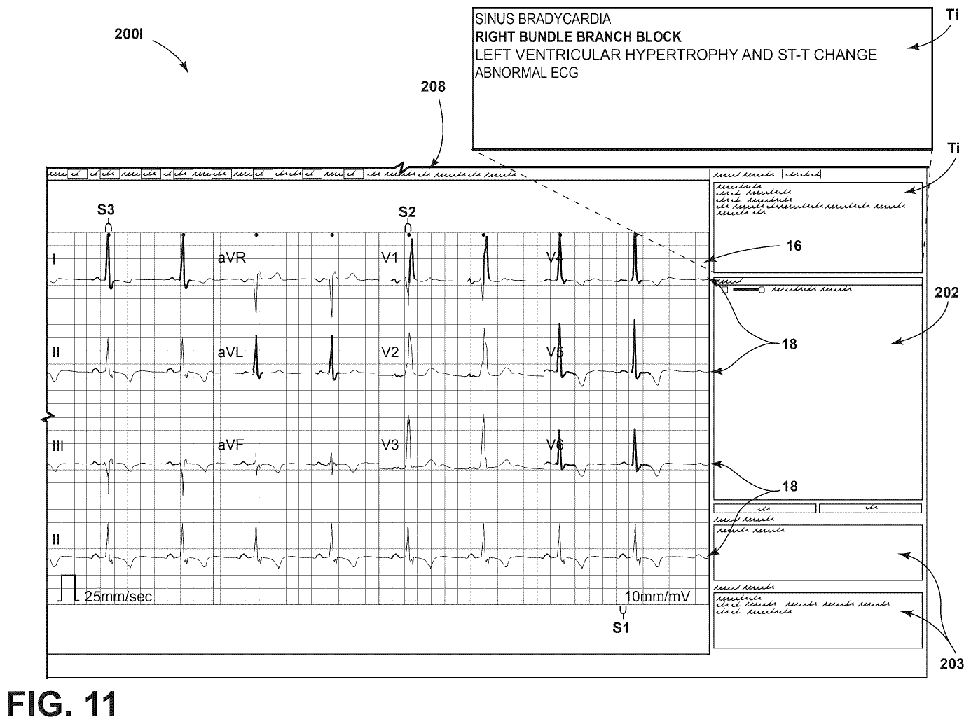

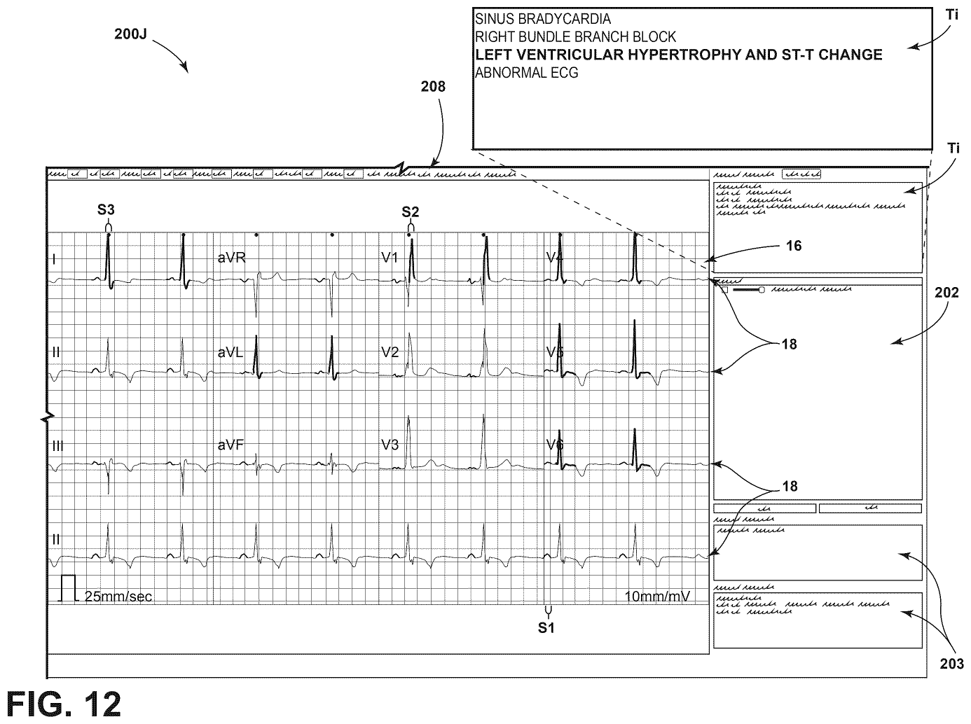

In FIG. 10 a second partial image 200H is illustrated with neither the textual information Ti or the segments selected (e.g., via the user interface 14). As a result, the segments S1-S3 and the textual information Ti are colored, but not enlarged. In FIG. 11, a third partial image 2001 is illustrated where the textual information Ti that recites the right bundle branch block or the segment S2 has been selected (e.g., via the user interface). As a result, the segment S2 of the waveform 18 has been enlarged (e.g., thickened). In FIG. 12, a fourth partial image 200J is illustrated where the textual information Ti that recites the left ventricular hypertrophy and ST-T change or the segment S3 has been selected (e.g., via the user interface). As a result, the segment S3 of the waveform 18 has been enlarged (e.g., thickened).

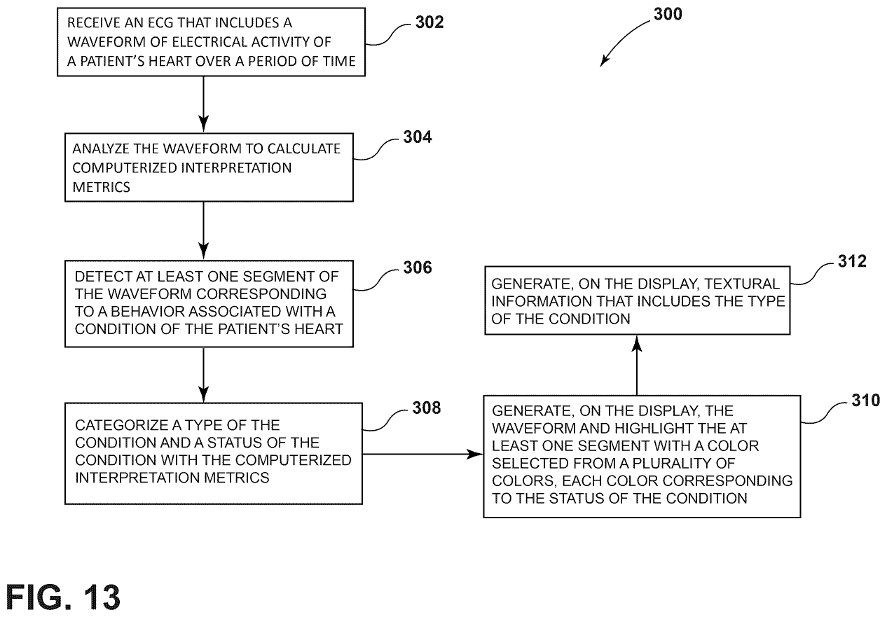

FIG. 13 shows a method 300 of visualizing a computerized interpretation on a display 12 with a user interface 14. At step 302, the method 300 includes receiving an ECG that includes a waveform of electrical activity of a patient's heart over a period of time (e.g., directly from the electrocardiogram device 20, a remote storage 150, and/or the like). At step 304, the method 300 includes analyzing the waveform to calculate computerized interpretation metrics. At step 306, the method 300 includes detecting at least one segment of the waveform corresponding to a behavior associated with a condition of the patient's heart. At step 308, the method 300 includes categorizing a type of condition or the status of the condition with the computerized interpretation metrics. At step 310, the method 300 includes generating, on the display 12, the waveform and highlight the at least one segment with a color selected from a plurality of colors, each color of the plurality of colors corresponding to the type of condition and/or the status of the condition. At step 312, the method 300 includes generating, on the display 12, textual information that includes the type of condition and/or the status of the condition.

The disclosure herein may be further summarized in the following paragraphs and further characterized by combinations of any and all of the various aspects described therein.

According to one aspect of the present disclosure, an electrocardiogram (“ECG”) interpretation system includes a display, a user interface, a processor, and a memory. The memory contains instructions that, when executed by the processor, cause the processor to receive an ECG that includes a waveform of electrical activity of a patient's heart over a period of time and analyze the waveform to calculate computerized interpretation metrics. The processor is further caused to detect at least one segment of the waveform corresponding to a behavior associated with a condition of the patient's heart and categorize a type of the condition and a status of the condition selected from a group including at least normal and abnormal with the computerized interpretation metrics. The processor is further caused to generate, on the display, the waveform and highlight the at least one segment with a color selected from a plurality of colors, each color of the plurality of colors corresponding to the type of condition or the status of the condition. The processor is further caused to generate, on the display, textual information that includes the type of condition or the status of the condition.

According to another aspect, textual information includes information related to portions of a waveform other than at least one segment.

According to still another aspect, textual information that includes a type of condition or a status of the condition is highlighted in a color of a highlight applied to at least one segment.

According to yet another aspect, a highlight on a waveform includes colorizing at least one segment in a color.

According to another aspect, a highlight on textual information includes colorizing a font in a color or a background behind the font in the color.

According to still yet another aspect, a user interface includes a cursor, and a processor is caused to detect if the cursor is aligned with textual information that includes a type of condition or a status of the condition, and enlarge at least one segment on a display.

According to yet another aspect, the at least one segment is thickened.

According to another aspect, the at least one segment zoomed in on.

According to still another aspect, a user interface includes a cursor, and a processor is caused to detect if the cursor is aligned with at least one segment, and enlarge textual information that includes a type of condition or a status of the condition.

According to yet another aspect, the textual information that includes the type of condition or the status of the condition is bolded.

According to another aspect, the textual information that includes the type of condition or the status of the condition is generated in larger font.

According to still another aspect, a user interface includes a keypad, and a processor is caused to generate a diagnosis section, receive comments from the keypad related to a clinical diagnosis, and generate the comments in the diagnosis section.

According to still yet another aspect, a plurality of colors includes a first color assigned to normal and a second color assigned to abnormal.

According to another aspect, a plurality of colors further includes a third color assigned to borderline.

According to still another aspect, a plurality of colors further includes a fourth color assigned to critical.

According to still another aspect, a plurality of colors further includes a fifth color assigned to abnormal rhythm.

According to another aspect of the present disclosure, an electrocardiogram (“ECG”) interpretation system includes a display, a user interface, a processor, and a memory. The memory contains instructions that, when executed by the processor, cause the processor to receive an ECG that includes a waveform of electrical activity of a patient's heart over a period of time and analyze the waveform to calculate computerized interpretation metrics. The processor is further caused to detect at least one segment of the waveform corresponding to an abnormality of the patient's heart with the computerized interpretation metrics. The processor is further caused to generate on the display, the waveform, and generate, on the display, textual information that includes the type of the abnormality and highlight the textual information with a color selected from a plurality of colors, each color of the plurality of colors corresponding to the severity of the abnormality.

According to another aspect, a processor is caused to highlight at least one segment with a color selected from a plurality of colors.

According to still another aspect, a highlight on a waveform includes colorizing at least one segment in a color.

According to still yet another aspect, a highlight on a textual information includes colorizing a font in a color.

According to another aspect, a user interface includes a cursor, and a processor is caused to detect if the cursor is aligned with textual information that includes a type of an abnormality and enlarge at least one segment on a display.

According to still another aspect, the at least one segment is thickened.

According to another aspect, the at least one segment is zoomed in on.

According to still another aspect, a user interface includes a cursor, and a processor is caused to detect if the cursor is aligned with at least one segment, and enlarge textual information that includes a type of an abnormality.

According to still yet another aspect, the textual information that includes the type of the abnormality is bolded.

According to another aspect, the textual information that includes the type of the abnormality is generated in larger font.

According to still another aspect, a user interface includes a keypad, and a processor is caused to generate a diagnosis section, receive comments from the keypad related to a clinical diagnosis, and generate the comments in the diagnosis section.

According to yet another aspect of the present disclosure, an electrocardiogram (“ECG”) interpretation system includes a display, a user interface, a processor, and a memory. The memory contains instructions that, when executed by the processor, cause the processor to receive an ECG that includes a waveform of electrical activity of a patient's heart over a period of time and analyze the waveform to calculate computerized interpretation metrics. The processor is further caused to detect at least one segment of the waveform corresponding to a behavior associated with a condition of the patient's heart and categorize a type of the condition and a status of the condition selected from a group including at least normal and abnormal with the computerized interpretation metrics. The processor is further caused to generate, on the display, the waveform and highlight the at least one segment with a color selected from a plurality of colors, each color of the plurality of colors corresponding to the type of condition or the status of the condition. The processor is further caused to generate, on the display, textual information that includes the type of condition or the status of the condition and highlight the textual information with the color selected from the plurality of colors, each color of the plurality of colors corresponding to the type of condition or the status of the condition.

According to another aspect, a highlight on a waveform includes colorizing at least one segment in a color.

According to still another aspect, a highlight on textual information includes colorizing a font in a color.

According to still yet another aspect of the present disclosure, an electrocardiogram (“ECG”) interpretation system includes a display, a user interface, a processor, and a memory. The memory contains instructions that, when executed by the processor, cause the processor to receive an ECG that includes a waveform of electrical activity of a patient's heart over a period of time and analyze the waveform to calculate computerized interpretation metrics. The processor is further caused to detect at least one segment of the waveform corresponding to a behavior associated with a condition of the patient's heart and categorize a type of the condition and a status of the condition selected from a group including at least normal and abnormal with the computerized interpretation metrics. The processor is further caused to generate, on the display, the waveform and highlight the at least one segment with a first color selected from a plurality of first colors, each first color corresponding to the type of condition or the status of the condition. The processor is further caused to generate, on the display, textual information that includes the type of condition or the status of the condition and highlight the textual information with the first color selected from the first plurality of colors, each color of the plurality of colors corresponding to the type of condition or the status of the condition.

According to another aspect, a processor is caused to highlight a background behind at least one segment with a second color selected from a plurality of second colors, each second color corresponding to a severity of an abnormality.

According to still another aspect, a processor is caused to highlight a background behind textual information with a second color selected from a plurality of second colors.

According to still another aspect of the present disclosure, an electrocardiogram (“ECG”) interpretation system includes an electrocardiogram device configured to place over a patient's heart that generates a cardiac signal. The ECG interpretation system further includes a display, a user interface, a processor, and a memory. The memory contains instructions that, when executed by the processor, cause the processor to receive, from the electrocardiogram device, the cardiac signal and generate an ECG that includes a waveform of electrical activity of a patient's heart over a period of time, and analyze the waveform to calculate computerized interpretation metrics. The processor is further caused to detect at least one segment of the waveform corresponding to a behavior associated with a condition of the patient's heart and categorize a type of the condition and a status of the condition selected from a group including at least normal and abnormal with the computerized interpretation metrics. The processor is further caused to generate, on the display, the waveform and highlight the at least one segment with a color selected from a plurality of colors, each color of the plurality of colors corresponding to the type of condition or the status of the condition. The processor is further caused to generate, on the display, textual information that includes the type of condition or the status of the condition.

According to still yet another aspect, a method of interpreting an electrocardiogram (“ECG”) includes receiving an ECG that includes a waveform of electrical activity of a patient's heart over a period of time. The waveform is analyzed to calculate computerized interpretation metrics and at least one segment of the waveform is detected that corresponds to a behavior associated with a condition of the patient's heart. The method further includes categorizing a type of the condition and a status of the condition selected from a group including at least normal and abnormal with the computerized interpretation metrics, and generating, on a display, the waveform and highlight the at least one segment with a color selected from a plurality of colors, each color of the plurality of colors corresponding to the type of condition or the status of the condition. On the display, textual information is generated that includes the type of the-condition and the status of the condition.

According to another aspect, textual information is highlighted that includes a type of a condition and a status of the condition with the color.

According to yet another aspect, at least one segment is colorized in a color.

According to still another aspect, a font of the textual information is colorized in a color.

According to still yet another aspect, a background behind a font of textual information is colorized in a color.

According to another aspect, at least one segment is thickened.

According to yet another aspect, at least one segment is zoomed in on.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the end-points of each of the ranges are significant both in relation to the other end-point, and independently of the other end-point.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. An electrocardiogram (“ECG”) interpretation system comprising:
a display;
a user interface; and
a processor and a memory, the memory containing instructions that when executed by the processor cause the processor to:
receive an ECG that includes a waveform of electrical activity of a patient's heart over a period of time;
analyze the waveform to calculate computerized interpretation metrics;
detect at least one segment of the waveform corresponding to a behavior associated with a condition associated with a condition of the patient's heart;
categorize a type of the condition and a status of the condition selected from a group including at least normal and abnormal with the computerized interpretation metrics;
generate, on the display, the waveform and highlight the at least one segment with a color selected from a plurality of colors, each color of the plurality of colors corresponding to the type of condition or the status of the condition; and
generate, on the display, textual information that includes the type of the condition and the status of the condition.

2. The ECG interpretation system of claim 1, wherein the textual information further includes information related to portions of the waveform other than the at least one segment.

3. The ECG interpretation system of claim 2, wherein the textual information that includes the type of the type of condition and is highlighted in the color of the highlight to the at least one segment.

4. The ECG interpretation system of claim 3, wherein the highlight on the waveform includes colorizing the at least one segment in the color.

5. The ECG interpretation system of claim 4, wherein the highlight on the textual information includes colorizing a font of the textual information in the color.

6. The ECG interpretation system of claim 4, wherein the highlight on the textual information includes colorizing a background behind a font of the textual information in the color.

7. The ECG interpretation system of claim 1, wherein the processor is further caused to:
detect if a user interacts with the textual information that includes the type of condition or the status of the condition; and
enlarge the at least one segment on the display.

8. The ECG interpretation system of claim 7, wherein the at least one segment is thickened.

9. The ECG interpretation system of claim 7, wherein the at least one segment zoomed in on.

10. The ECG interpretation system of claim 1, wherein the processor is further caused to:
detect if a user interacts with the at least one segment; and
enlarge the textual information that includes the type of condition or the status of the condition.

11. The ECG interpretation system of claim 10, wherein the textual information that includes the type of condition also includes the status of the condition.

12. The ECG interpretation system of claim 10, wherein the textual information that includes the type of condition and the status of the condition is generated in larger font.

13. The ECG interpretation system of claim 1, wherein the user interface includes a keypad, and wherein the processor is further caused to:
generate a diagnosis section;
receive comments from the keypad related to a clinical diagnosis; and
generate the comments in the diagnosis section.

14. An electrocardiogram (“ECG”) interpretation system comprising:
a display;
a user interface; and
a processor and a memory, the memory containing instructions that when executed by the processor cause the processor to:
receive an ECG that includes a waveform of electrical activity of a patient's heart over a period of time;
analyze the waveform to calculate computerized interpretation metrics;
detect at least one segment of the waveform corresponding to a behavior associated with a condition of the patient's heart;
categorize a type of the condition and a status of the condition selected from a group including at least normal and abnormal with the computerized interpretation metrics;
generate, on the display, the waveform; and
generate, on the display, textual information that includes the type of the abnormality and highlight the textual information with a color selected from a plurality of colors, each color of the plurality of colors corresponding to the severity of the abnormality.

15. The ECG interpretation system of claim 14, wherein the processor is further caused to highlight the at least one segment with the color selected from a plurality of colors.

16. The ECG interpretation system of claim 15, wherein the highlight on the waveform includes colorizing the at least one segment in the color.

17. The ECG interpretation as in claim 16, wherein the user interface includes a cursor, and wherein the processor is further caused to:
detect if the cursor is aligned with the at least one segment; and
enlarge the textual information that includes the type of the abnormality.

18. The ECG interpretation system of claim 17, wherein the textual information that includes the type of the abnormality is bolded.

19. A method of interpreting an electrocardiogram (“ECG”) comprising:
receiving an ECG that includes a waveform of electrical activity of a patient's heart over a period of time;
analyzing the waveform to calculate computerized interpretation metrics;
detecting at least one segment of the waveform corresponding to a behavior associated with a condition of the patient's heart;
categorizing a type of the condition and a status of the condition selected from a group including at least normal and abnormal with the computerized interpretation metrics;
generating, on a display, the waveform and highlight the at least one segment with a color selected from a plurality of colors, each color of the plurality of colors corresponding to the type of condition or the status of the condition; and
generating, on the display, textual information that includes the type of the condition and the status of the condition.

20. The method of claim 19, further including highlighting the textual information that includes the type of the condition and the status of the condition with the color and colorizing the at least one segment in the color.
